(19) United States
(12) Patent Application Publication
(10) Pub. No.: US 2025/0041304 A1
(43) Pub. Date: Feb. 6, 2025

(54) Treatment of Hand Eczema with Baricitinib

(71) Applicant: Eli Lilly and Company, Indianapolis, IN (US)

(72) Inventors: Susanne GROND, Vienna (AT); Elisabeth RIEDL, Vienna (AT)

(21) Appl. No.: 18/292,486

(22) PCT Filed: Jul. 29, 2022
(86) PCT No.: PCT/US2022/038744

§ 371 Date: Jan. 26, 2024

Related U.S. Application Data
(60) Provisional application No. 63/203,757, filed on Jul. 30, 2021.

(51) Int. Cl. A61K 31/519; A61K 9/20

(52) U.S. Cl. CPC A61K 31/519; A61K 9/20

(57) ABSTRACT

Methods of treating Hand Eczema with baricitinib, including formulations and dose regimens. The amount of baricitinib may be administered as a 4 mg tablet or pill that includes one or more excipients. The amount of baricitinib may be administered daily or at some other frequency.

The present invention relates to the field of medicine. More particularly, the present invention relates to the treatment of patients with Moderate to Severe Atopic Hand Eczema (HE)

Baricitinib is an approved medication that belongs to the pharmacological class of Janus kinase (JAK) inhibitors. Janus kinases are a family of four (4) protein tyrosine kinases (JAK1, JAK2, JAK3, and tyrosine kinase 2 [TYK2]) that play a role in cytokine signal transduction. Baricitinib demonstrates selectivity for, and inhibition of, JAK1 and JAK2 with lower potency towards inhibition of JAK3 or TYK2 (Fridman J S, et al. 2010, “Selective inhibition of JAK1 and JAK2 is efficacious in rodent models of arthritis: preclinical characterization of INCB028050,” J Immunol. 2010; 184(9):5298-5307.) In isolated enzyme assays, baricitinib inhibited the activities of JAK1, JAK2, TYK2, and JAK3 with half-maximal inhibitory concentration values of 5.9, 5.7, 53, and >400 nM, respectively (See id.) Janus kinases are enzymes that transduce intracellular signals from cell surface receptors for a number of cytokines and growth factors involved in hematopoiesis, inflammation, and immune function (e.g., interleukin [IL]-2, IL-6, IL-12, IL-15, IL-23, interferons, and granulocyte-macrophage colony-stimulating factor signal through the JAK family). (O'Shea et al., “The JAK-STAT pathway: impact on human disease and therapeutic intervention,” Annu Rev Med. 2015; 66:311-28.) Within the intracellular signaling pathway, JAKs phosphorylate and activate signal transducers and activators of transcription (STATs), which activate gene expression within the cell. Baricitinib modulates these signaling pathways by partially inhibiting JAK1 and JAK2 enzymatic activity, reducing the phosphorylation and activation of STATs and reducing inflammation, cellular activation, and proliferation of key immune cells. (O'Shea et al., “JAKs and STATs in immunity, immunodeficiency, and cancer,” N Engl J Med. Review 2013 Jan. 10; 368(2):161-70.)

Atopic dermatitis (AD), also known as atopic eczema, is a common, chronic, relapsing, and highly symptomatic inflammatory skin disease. Patients with AD may have skin lesions that can be acute, presenting as oozing, crusted, eroded vesicles, papules, or erythematous plaques. Patients may also present with subacute skin changes, characterized by thick and excoriated plaques, or chronic lesions, with lichenified, slightly pigmented, excoriated plaques (Bieber 2010). Disease severity can be mild, moderate, or severe, depending on the degree of skin inflammation. In clinical practice, additional features such as the location of skin lesions and the impact of itch on sleep and overall quality of life (QoL) are taken into consideration when assessing the severity of AD. The location of skin lesions is considered critical, especially when an exposed and functionally important body area like the hands are affected. Baricitinib has been tested in an extensive clinical development program across multiple indications and is currently approved in Europe for the treatment of rheumatoid arthritis (RA) and moderate-to-severe AD in adult patients who are candidates for systemic therapy.

Atopic hand eczema (also known as atopic hand dermatitis) is a subtype of HE that occurs in patients with AD. In line with AD in other body areas, atopic HE can be classified as mild, moderate, or severe. Given the central role that hands play in everyday functional activities, atopic HE is associated with physical and psychological impairment and has a substantial psychosocial, workforce, and economic impact (Veien et al. 2008). Due to the underlying skin barrier defect in AD, the course of atopic HE is usually highly chronic, characterized by recurrent flares, and often refractory to treatment. Secondary mechanical factors due to the exposed body site further complicate the course of the disease. Treatment options for atopic HE patients are very limited and hampered by unsatisfying efficacy and low tolerability of available systemic therapies. Trigger factors for HE include environmental exposures such as cold or dry weather conditions and humidity, and occupational factors including wet work, irritants, and exposure to direct allergens and mechanical irritations. Thus, “trigger avoidance” is usually the first therapeutic action for patients with HE, but many people find that to be unavoidable or unsatisfactory, as they cannot reduce the environmental or work-related trigger factors.

In patients with mild-to-moderate HE, treatment options are limited to topical therapies, mainly topical corticosteroids (TCS), and to phototherapy. These therapies are usually insufficient to control severe, chronic HE, which warrant systemic treatment independent of body surface area (BSA) involvement, given the functional impairments that occur when hand use is restricted by chronic HE. Systemic treatment options for chronic HE are very limited. Available systemic treatments are associated with unsatisfactory outcomes and side effects, which results in a poor benefit/risk profile. Currently, the only approved systemic therapy for chronic HE in Europe is alitretinoin, which is associated with known retinoid side effects, especially during long-term treatment, and therefore is only prescribed as a last resort. Furthermore, in patients with concurrent AD, this medication is not advisable due to its effects on the skin barrier and skin microbiome.

Thus, there are still large segments of the patient population for which there is no or insufficient treatment options for HE. This is especially true for people who use their hands in their jobs. Such people may not achieve a HECSI decrease of 75% (as will be described herein). For these and other reasons, there exists an unmet need for an improved treatment of HE. Such treatment should address the autoimmune cause of HE and, preferably, prevent or treat the autoimmune response that causes HE. Additionally, such treatment for HE should treat the people whose work or environment trigger HE and be able to treat chronic HE. Further, the treatment should address the itch (pruritus), and/or pain associated with HE. As with all therapeutic treatments, safety and toxicity remain a limitation, thus any improved treatments must not be attendant on unacceptable safety and toxicity profiles and provide a clinical benefit.

The present invention provides a therapeutic treatment for the treatment of HE that overcomes one or more of the challenges recognized above.

In some embodiments, a method of treating a patient in need of treatment HE is provided comprising administering to said patient an amount of baricitinib, or a pharmaceutically acceptable salt thereof, or a pharmaceutical formulation thereof. In some embodiments, the amount of baricitinib is administered orally. For example, the oral administration may comprise giving the patient a tablet that includes one or more excipients. In further embodiments, said pill comprises 4 mg of baricitinib, although other amounts of baricitinib may also be used.

Further embodiments comprise a method of treating a patient in need of treatment of one of HE by administering to said patient an amount of baricitinib, or a pharmaceutical formulation thereof, wherein the patient's HECSI score is assessed at Day 0 and then treatment with baricitinib is administered, and then the patient's HECSI score is re-assessed (such as, for example, after 16 weeks and 32 weeks, although other times may be used). In some embodiments, after the HECSI score is re-assessed, the patient's HECSI score has decreased. Patients may be assessed for their HECSI score, weekly, every two weeks, or monthly, etc. In further embodiments, the HECSI score is re-assessed before, during or after Week 16 of treatment with baricitinib. During some embodiments, this treatment involves administering baricitinib in a daily dose (at, for example, 4 mg or some other dose).

In further embodiments, the patient's HECSI score may indicate that he or she should end therapy prior to 16 or 32 weeks. In other words, the doctor may determine, after looking at the patient after a certain period of time (such as 2 weeks, or 4 weeks, or 6 weeks, or 8 weeks, or 10 weeks, or 12 weeks, or 14 weeks, or 18 weeks, or 20 weeks, or 22 weeks, or 24 weeks, or 26 weeks, or 28 weeks, or 30 weeks) that the patient's HE (as measured by the HECSI score or some other metric) has improved such that he or she may discontinue therapy. In other embodiments, the patient and doctor may continue on receiving treatment (baricitinib) after 32 weeks (such as for an additional 16 weeks, and additional 32 weeks, and additional 48 or 64 weeks) or an indeterminate period of time (or some other period of time), as determined by the patient and/or his/her doctor.

Embodiments may also comprise a method of treating a patient in need of treatment of one of HE by administering to said patient an amount of baricitinib, or a pharmaceutical formulation thereof, wherein the patient's HECSI score is assessed at Day 0 and then treatment with baricitinib is administered, and then the patient's HECSI score is re-assessed. In some embodiments, after the HECSI score is re-assessed, the patient's HESCI has decreased (such as, for example, at least a 75% decrease). In some of these embodiments, the HECSI is re-assessed before, during or after Week 16 of treatment with baricitinib.

Other embodiments provide the use of baricitinib, or a pharmaceutical formulation thereof in the manufacture of a medicament for the treatment of at least one of HE. In many of these embodiments, the amount of the baricitinib is 4 mg. In other embodiments, the baricitinib is administered in the form of a pill.

Furthermore, the present invention provides baricitinib, or a pharmaceutical formulation comprising baricitinib, for use in treating HE. In some embodiments, the baricitinib, or a pharmaceutical formulation comprising baricitinib, is in the form of a pill that includes one or more excipients.

The present embodiments relate to the use of baricitinib in the manufacture of a medicament for the treatment of HE. This use may have the baricitinib in the form of a pill that includes one or more excipients. The use may also be such that the patient's HECSI score is assessed at Day 0 and re-assessed following administration of baricitinib, such as, for example, at Week 16. The use may further be such that the baricitinib is administered daily, such as, for example, in a 4 mg daily dose.

Baricitinib is a Janus kinase (JAK) inhibitor (and more specifically a selective JAK 1 and JAK 2 inhibitor) with the chemical name {1-(ethylsulfonyl)-3-[4-(7H-pyrrolo[2,3-d]pyrimidin-4-yl)-1H-pyrazol-1-yl]azetidin-3-yl}acetonitrile. Baricitinib has the following structural formula:

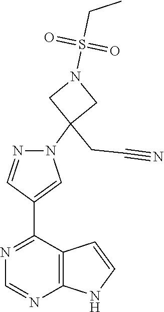 [SMILES: CCS(=O)(=O)N1CC(CC#N)(n2cc(-c3ncnc4[nH]ccc34)cn2)C1]

Additional information about baricitinib including methods of making the compound may be found in U.S. Pat. Nos. 8,158,616 and 8,420,629. Additional methods for making baricitinib are found in U.S. Patent Application Publication No. 2018/0134713.

Baricitinib is a known medicine that is approved in the United States and Europe (and other countries) for the treatment of rheumatoid arthritis and is commercially available under the trademark OLUMIANT®. The European Medicines Agency has also approved baricitinib for treatment of moderate to severe atopic dermatitis.

In some jurisdictions, OLUMIANT® is available in pill form, wherein the pill includes a designated amount of baricitinib and the following excipients: croscarmellose sodium, magnesium stearate, mannitol, microcrystalline cellulose, ferric oxide, lecithin (soya), polyethylene glycol, polyvinyl alcohol, talc and titanium dioxide. In preferred embodiments of the present invention, the amount of baricitinib that is used to treat the patient is administered by giving the patient one or more pills of OLUMIANT®. Of course, other dosages forms, pharmaceutical compositions of baricitinib, etc. may also be used.

Those skilled in the art will also appreciate that, in other embodiments, a pharmaceutically acceptable salt of baricitinib may be used. Pharmaceutically acceptable salts are known. As used herein, the term “pharmaceutically acceptable salt” refers to derivatives of the compounds herein, where a compound herein is modified by making acid or base salts thereof. Pharmaceutically acceptable salts, and processes for preparing the same, are well known in the art (see, e.g., Remington: The Science and Practice of Pharmacy, L. V. Allen, Ed., 22^{nd} Edition, Pharmaceutical Press, 2012). By way of example, pharmaceutically acceptable salts include, but are not limited to, mineral or organic acid salts of basic residues such as amines, or alkali or organic salts of acidic residues such as carboxylic acids. Pharmaceutically acceptable salts include the conventional non-toxic salts or the quaternary ammonium salts of a compound herein formed, for example, from non-toxic inorganic or organic acids. Such conventional nontoxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. Pharmaceutically acceptable salts are those forms of a compound herein, suitable for use in contact with the tissues of human beings and animals without excessive toxicity, irritation, allergic response, or other problem or complication, commensurate with a reasonable benefit/risk ratio. Pharmaceutically acceptable salt forms of a compound herein can be synthesized to contain a basic or acidic moiety by conventional chemical methods. Generally, such salts are, for example, prepared by reacting the free acid or base forms of the compound with a stoichiometric amount of the appropriate base or acid in water or in an organic solvent, or in a mixture of the two; generally, nonaqueous media like ether, ethyl acetate, ethanol, isopropanol, or acetonitrile are preferred (see, e.g., Stahl et al., “Handbook of Pharmaceutical Salts: Properties, Selection and Use” (Wiley-VCH 2^{nd} ed. 2011)).

As referred to herein and as generally known in the art, the term “dose” refers to an amount of baricitinib that is administered to a subject. A “dose regimen” or “dosage regimen” as generally known in the field and as may be referred to interchangeably herein includes a treatment schedule for administering a set (i.e., series or sequence) of doses to be administered to a patient over a period of time. The present invention includes a dose regimen for the HE treatments of the present invention. Specifically, prior to the day in which the patient receives treatment with baricitinib, (which is referred to as “Day 0”) the patient's condition is assessed. This assessment of the patient's HE conditions may occur 5 weeks prior to Day 0 or in other embodiments 8-35 days before treatment. In some embodiments, the patient's HE conditions may be assessed 2, or 3 or 4 weeks prior to Day 0, and then again 1 week prior to Day 0. Other times when the assessment of the patient's HE conditions is done may also be used. Then on Day 0, the assessments may be repeated, and this data will be the “baseline” for further comparison. Such assessments of the patient (and his/her HE and AD condition) may involve determining one or more of the following scores or measurements (each of these scores are described herein): the patient's HECSI score;
the patient's EASI score;
the patient's itch and skin pain NRS score;
the vIGA-AD score;
the patient's HADS score;
the patient's DLQI score;
the patient's QOLHEQ score;
the patient's mTLSS score;
the patient's WPAI score;
the ADSS score;
a score based upon the photographic guide for assessing severity of chronic HE; and/or
any additional patient related outcomes and/or patient provided information relevant to HE.

In addition to the assessment of the patient's HE condition (in the manner outlined above) that occurs before treatment and on Day 0, similar assessments may occur after Day 0, e.g., after the patient receives baricitinib. Such assessments may occur weekly, or every two weeks, every three or four weeks, as desired. In some embodiments, the treatment with baricitinib will last 16 weeks or 32 weeks, and the skilled practitioner will appreciate how often s/he should assess the patient's HE condition while the patient is receiving baricitinib. In many embodiments, the assessments will track how the patient has improved in the various scores at or after 16 weeks and/or at or after 32 weeks.

In some embodiments, patients will stop using any systemic treatments that are used for HE about 4 weeks prior to Day 0. Such treatments could include cyclosporin or a systemic steroid (such as prednisone). The patient may also stop taking any TCS treatments 1 week before Day 0. In some embodiments the patient may receive TCS starting on Day 0 and during the treatment with baricitinib.

At Day 0, the patient will begin to receive treatment with baricitinib. According to some embodiments, this administration of baricitinib may occur daily (or at some other specified dosing time period) and be at the dose of 4 mg of baricitinib (such as, for example by directing the patient to administer a 4 mg pill of baricitinib). In some of the presently preferred embodiments, each day throughout a 16-week period, the patient is directed to take a daily dose of baricitinib. The amount of baricitinib may vary. In some embodiments, the patient takes 4 mg per day while in other embodiments, different doses of baricitinib (e.g., 2 mg, 8 mg, 12 mg, 16 mg, 20 mg, etc.) are given as determined by the patient and/or his or her physician. The baricitinib may be administered once a day, or twice a day, three times a day, four times a day, etc. as needed. Those skilled in the art will appreciate how to determine the appropriate dosing intervals, as needed, if the selected dosing regimen involves administering more than one dose during a single day.

One of the key measurements that will be assessed in order to determine the patient's condition is the HECSI (Hand Eczema Severity Index) score. As noted herein, the baseline HECSI score may be obtained on Day 0, before the patient receives baricitinib. Also, days or weeks before Day 0, the patient's HECSI score may also be obtained, as this may provide additional information. The patient's HECSI will be systematically measured at the time points noted herein. In some embodiments, the patients will see (or a majority of patients will see) at least a 75% improvement in the patient's HECSI score at week 16. This 75% improvement from the baseline HECSI score is sometimes referred to as HECSI75. In other embodiments, the patients will see (or a majority of patients will see) at least a 75% improvement in the patient's HECSI score at week 32. Of course, other improvements in the HECSI score, such as 25%, 30%, 35%, 40%, 45%, 50% 55%, 60%, 65% 70%, 80%, 85%, 90%, 95% or greater than 95% are also possible.

The HECSI score is an objective validated clinical scale that assesses disease severity of hand eczema based on clinical signs. This is a validated scoring system with excellent agreement for both interobserver and intra-observer reliability. Similar to the EASI scale (discussed below) that is used to assess overall disease severity in AD by a qualitative and quantitative evaluation of skin inflammation, the HECSI looks qualitatively and quantitatively at skin inflammation of the hands and wrists. Reflecting the typical morphological skin changes in AD in other body areas (erythema, oozing, vesiculation, dryness, papulation/infiltration, and scaling), the HECSI assesses the following 6 signs of HE that overlap with the typical changes of AD lesions, as assessed by EASI, with the exception of fissures, linear deep and painful ulceration that contribute to the high burden of HE: erythema (redness), infiltration/papulation, vesicles, fissures, scaling, and oedema. These signs are assessed at 5 locations on the hands: fingertips, fingers (except tips), palm of hand, back of hands, and wrists. The extent of the lesions is taken into consideration when calculating the HECSI score. The total HECSI score ranges from 0 to 360 and is calculated by multiplying the score given for each location by the total sum of the intensity of each clinical feature. This score is calculated by multiplying the severity index (rated as 0 None, 1 Mild, 2 Moderate, and 3 Severe) by the surface area of the affected area to obtain the composite score.

The HECSI was validated initially in 2005 by Held and colleagues (Held E, Skoet R, Johansen J D, Agner T. The hand eczema severity index (HECSI): a scoring system for clinical assessment of hand eczema. A study of inter- and intraobserver reliability. Br J Dermatol. 2005; 152(2):302-307). The HECSI assessment is comparable to that of the EASI, which was verified in a similar study design in 2001 (Hanifin J M, Thurston M, Omoto M, et al.; EASI Evaluator Group. The eczema area and severity index (EASI): assessment of reliability in atopic dermatitis. Exp Dermatol. 2001; 10(1):11-18.) For the HECSI validation in 2005, a total of 15 patients suffering from HE were enrolled in the study along with 12 dermatologists as observers. Evaluating inter and intraobserver reliability, it was shown that there was overall good to excellent interobserver reliability for total HECSI scores and for the separate items in the assessment. The intraobserver reliability was also found to be acceptable.

The HECSI score has been employed in several studies carried out on patients with HE since it was validated:
Lerbaek A, Kyvik K O, Ravn H, et al. Clinical characteristics and consequences of hand eczema—an 8-year follow-up study of a population-based twin cohort. Contact Dermatitis 2008; 58(4):210-216;
Bauer A, Lange N, Matterne U, et al. Efficacy of pimecrolimus 1% cream in the long term management of atopic hand dermatitis. A double-blind RCT. J Dtsch Dermatol Ges. 2012; 10(6):426-433;
van Gils R F, Boot C R, Knol D L, et al. The effectiveness of integrated care for patients with hand eczema: results of a randomized, controlled trial. Contact Dermatitis. 2012; 66(4):197-204;
Charan U P, Peter C V D, Pulimood S A. Impact of hand eczema severity on quality of life. Indian Dermatol Online J. 2013; 4(2):102-105;
Oosterhaven J A F, Voorberg A N, Romeijn G L, et al. Effect of dupilumab on hand eczema in patients with atopic dermatitis: an observational study. J Dermatol. 2019; 46(8):680-685; and
Worm M, Bauer A, Elsner P, et al. Efficacy and safety of topical delgocitinib in patients with chronic hand eczema: data from a randomized, double-blind, vehicle-controlled phase II a study. Br J Dermatol. 2020; 182(5):1103-1110.

Recently, the responsiveness and interpretability of the HECSI score was examined (Oosterhaven J A F, Schuttelaar M L A. Responsiveness and interpretability of the Hand Eczema Severity Index. Br J Dermatol. 2020; 182(4), 932-939.) By scoring a total of 294 patients with HE at baseline and again after 4 to 12 weeks, a good responsiveness of the HECSI score was confirmed. Using the “photographic guide for assessing severity of chronic hand dermatitis” as anchor, Oosterhaven and Schuttelaar (2020) defined the severity grading as follows:
clear, 0
almost clear, 1-16
moderate, 17-37
severe, 38-116
very severe, greater than or equal to 117.
   (Oosterhaven J A F, Schuttelaar M L A. Responsiveness and interpretability of the Hand Eczema Severity Index. Br J Dermatol. 2020; 182(4), 932-939.)

The HECSI75, corresponds to greater than or equal to 75% improvement in hand area and severity. This score is comparable to the EASI75, which has been commonly considered to represent a clinically significant improvement in the severity and extent of A D (Schram M E, Spuls P I, Leeflang M M, et al. EASI, (objective) SCORAD and POEM for atopic eczema: responsiveness and minimal clinically important difference. Allergy. 2012; 67(1):99-106) in clinical trials. While examining the responsiveness and interpretability of the HECSI recently, Oosterhaven and Schuttelaar (Oosterhaven J A F, Schuttelaar M L A. Responsiveness and interpretability of the Hand Eczema Severity Index. Br J Dermatol. 2020; 182(4), 932-939) suggested that HECSI75 is necessary to reflect a true clinical improvement for the patient. HECSI75 was also chosen as the primary cutoff point in a recent study that examined the effect of dupilumab on HE (Oosterhaven J A F, Voorberg A N, Romeijn G L, et al. Effect of dupilumab on hand eczema in patients with atopic dermatitis: an observational study. J Dermatol. 2019; 46(8):680-685.)

In addition to the HECSI score, other scores may be obtained in the assessments of the atopic HE. In some embodiments, the mTLSS score is obtained as part of the patient's assessments of HE conditions (which may occur prior to Day 0, on Day 0, throughout the trial, at Week 16 and/or Week 32). mTLSS refers to the “Modified Total Lesion Symptom Score”. The mTLSS combines the evaluation of HE lesion severity (erythema, oedema, desquamation, fissures, hyperkeratosis/lichenification, vesicles) with the intensity of pruritus/pain. (See Bissonnette R, Diepgen T L, Elsner P, et al. Redefining treatment options in chronic hand eczema (CHE). J Eur Aca Dermatol Venereol. 2010; 24(suppl 3):1-20). This composite score assigns 0 (mild) to 3 (severe) to each component, giving a maximum disease severity of 21. The mTLSS score has been used as secondary endpoint in studies investigating alitretinoin in HE (Fowler J F, Graff O, Hamedani A G. A phase 3, randomized, double-blind, placebo-controlled study evaluating the efficacy and safety of alitretinoin (BAL4079) in the treatment of severe chronic hand eczema refractory to potent topical corticosteroid therapy. J Drugs Dermatol. 2014; 13(10):1198-1204.)

In some embodiments, the mean change of the mTLSS score will be assessed versus the baseline. In some embodiments, this will involve a change between 2-20 in the mTLSS score. In other embodiments, the change in the mTLSS score may be between 2-15. In other embodiments, the change in the mTLSS score may be between 2-12. In other embodiments, the change in the mTLSS score may be between 2-10 In other embodiments, the change in the mTLSS score may be between 2-8. In other embodiments, the change in the mTLSS score may be between 2-6. In other embodiments, the change in the mTLSS score may be between 2-4.

A score based upon the photographic guide for assessing severity of chronic hand dermatitis may be obtained as part of the patient's assessments of HE conditions as outlined herein. The Photographic guide is a validated global instrument that measures the morphological severity of HE on a 5-point scale (0=clear, 1=almost clear, 2=moderate; 3=severe; 4=very severe) using photographs representing the severity categories. The photographic guide has been shown to have a high level of interrater reliability and test-retest reproducibility (Coenraads P J, Van Der Walle H, Thestrup-Pedersen K, et al. Construction and validation of a photographic guide for assessing severity of chronic hand dermatitis. Br J Dermatol. 2005; 152(2):296-301). The photographic guide was also used as an anchor to define HECSI minimally important change and HECSI severity categories (Oosterhaven J A F, Schuttelaar M L A. Responsiveness and interpretability of the Hand Eczema Severity Index. Br J Dermatol. 2020; 182(4), 932-939).

In some embodiments, the photographic guide may give the patient a scale of 0 or 1 at week 16. In some embodiments, the photographic guide may give the patient a scale of 0 or 1 at week 32. In some embodiments, the photographic guide may give the patient a scale of 0 or 1 at week 4, 8 or 12. In some embodiments, the photographic guide may give the patient a scale of 0 or 1 at week 20, 24 or 28. In some embodiments, the photographic guide may give the patient a scale of 0 or 1 at time greater than week 16 or greater than week 32.

The EASI score (Eczema Area and Severity Index) may also be obtained as part of the AD assessments. The EASI assesses extent of disease at 4 body regions and measures 4 clinical signs including erythema, induration/papulation, excoriation, and lichenification each on a scale of 0 to 3. The EASI confers a maximum score of 72. The EASI evaluates 2 dimensions of AD: disease extent and clinical signs (Hanifin J M, Thurston M, Omoto M, et al.; EASI Evaluator Group. The eczema area and severity index (EASI): assessment of reliability in atopic dermatitis. Exp Dermatol. 2001; 10(1):11-18.) As is known in the art, the EASI score assigns proportionate body surface areas to four regions (10% to head/neck; 30% to trunk; 20% to upper extremities; and 40% to lower extremities) as well as a score of 0 None, 1 Mild, 2 Moderate, and 3 Severe. Then using the multiplication of the percentages noted above associated with the body surface area with the severity, a numeric score is obtained.

In some embodiments, the mean change of the EASI score will be assessed versus the baseline. In some embodiments, this will involve a change between 2-20 in the EASI score. In other embodiments, the change in the EASI score may be between 2-15. In other embodiments, the change in the EASI score may be between 2-12. In other embodiments, the change in the EASI score may be between 2-10 In other embodiments, the change in the EASI score may be between 2-8. In other embodiments, the change in the EASI score may be between 2-6. In other embodiments, the change in the EASI score may be between 2-4.

The vIGA-AD (Validated Investigator's Global Assessment of Atopic Dermatitis) score may also be obtained as part of the AD assessments. The vIGA-AD measures the investigator's global assessment of the patient's overall severity of their AD, based on a static, numeric 5-point scale from 0 (clear skin) to 4 (severe disease). The score is based on an overall assessment of the degree of erythema, papulation/induration, oozing/crusting, and lichenification (Simpson E, Bissonnette R, Eichenfield L F, et al. The validated Investigator Global Assessment for Atopic Dermatitis (vIGA-AD™): the development and reliability testing of a novel clinical outcome measurement instrument for the severity of atopic dermatitis. J Am Acad Dermatol. 2020; 83(3):839-846.)

In some embodiments, the mean change of the vIGA-AD score will be assessed versus the baseline. In some embodiments, this will involve a change between 2-20 in the vIGA-AD score. In other embodiments, the change in the vIGA-AD score may be between 2-15. In other embodiments, the change in the vIGA-AD score may be between 2-12. In other embodiments, the change in the vIGA-AD score may be between 2-10 In other embodiments, the change in the vIGA-AD score may be between 2-8. In other embodiments, the change in the vIGA-AD score may be between 2-6. In other embodiments, the change in the vIGA-AD score may be between 2-4.

Key symptoms of AD and HE, such as pruritus, sleep disturbance, skin pain, and interference with activities comprise an important aspect of the disease and the meaningfulness of clinical improvements for health related quality of life (HRQoL) can be assessed only by patients. Accordingly, in line with this, European bodies such as Germany's Institute for Quality and Efficiency in Health Care (IQWiG) and the UK's National Institute for Health and Care Excellence (NICE) have an increasing focus on HRQoL data, as assessed by patient reported outcome (PRO) measures, when evaluating treatment benefits for patients. Thus, the following assessments may also be made as part of the assessments described herein, including assessing clinically relevant improvements in itch, skin pain, sleep disturbance, and quality-of-life, as well as benefits on functional and psychological aspects of the disease, which pose unique social and therapeutic challenges for patients with HE.

Such PRO measurements include the Quality of Life in Hand Eczema Questionnaire (QOLHEQ) that assesses HE-specific impairment of HRQoL (Ofenloch R, Diepgen T, Weisshaar E, Apfelbacher C. The Quality of Life in hand eczema questionnaire: validation of a new assessment instrument. Das Gesundheitswesen. 2013; 75(08/09):A233.) It is a patient administered instrument composed of 30 questions relating to symptoms, emotions, functioning, treatment, and prevention. It has been validated for national use in Japan (Minamoto K, Diepgen T L, Sato K, et al. Quality of Life in Hand Eczema Questionnaire: Validation of the Japanese version of a disease-specific measure of quality of life for hand eczema patients. J Dermatol. 2018; 45(11):1301-1305), Germany ((Ofenloch R, Diepgen T, Weisshaar E, Apfelbacher C. The Quality of Life in hand eczema questionnaire: validation of a new assessment instrument. Das Gesundheitswesen. 2013; 75(08/09):A233), and the Netherlands (Oosterhaven J A F, Ofenloch R F, Schuttelaar M L A. Validation of the Dutch quality of life in hand eczema questionnaire (QOLHEQ). Br J Dermatol. 2020b; 183(1):86-95), showing that it is a valid, reliable, and responsive measure of HRQoL in patients with HE, and has furthermore undergone cross-cultural validation (Ofenloch R F, Oosterhaven J A, Susitaival P, et al. Cross-cultural validation of the Quality of Life in Hand Eczema Questionnaire (QOLHEQ). J Invest Dermatol. 2017; 137(7):1454-1460). A recent interpretability study has defined greater than or equal to 22 points as minimally important change and has proposed to incorporate the QOLHEQ into a core outcome set for HE (Ofenloch R F, Weisshaar E, Apfelbacher C. New evidence for construct validity and interpretability of the German Quality of Life in Hand Eczema Questionnaire (QOLHEQ) Contact Dermatitis. 2020; 83(3):189-195; Oosterhaven J A F, Ofenloch R F, Schuttelaar M L A. Interpretability of the Quality Of Life in Hand Eczema Questionnaire. J Invest Dermatol. 2020a; 140(4):785-790. et al. 2020a).

In some embodiments, the mean change of the QOLHEQ score will be assessed versus the baseline at weeks 16, 32 or at other time periods. In some embodiments, this will involve a change between 2-20 in the QOLHEQ score. In other embodiments, the change in the QOLHEQ score may be between 2-15. In other embodiments, the change in the QOLHEQ score may be between 2-12. In other embodiments, the change in the QOLHEQ score may be between 2-10 In other embodiments, the change in the QOLHEQ score may be between 2-8. In other embodiments, the change in the QOLHEQ score may be between 2-6. In other embodiments, the change in the QOLHEQ score may be between 2-4.

Such PRO measurements may also include the Dermatology Life Quality Index (DLQI) score. It is a 10-item, validated, quality-of-life questionnaire that covers 6 domains including symptoms and feelings, daily activities, leisure, work and school, personal relationships, and treatment. Scores range from 0 to 30, with higher scores indicating greater impairment of QoL. A DLQI total score of 0 to 1 is considered as having no effect on a patient's health-related QoL (Hongbo Y, Thomas C L, Harrison M A, et al. Translating the science of quality of life into practice: what do dermatology life quality index scores mean? J Invest Dermatol. 2005; 125(4):659-664) and a 4-point change from baseline is considered as the minimal clinically important difference threshold (Khilji F. Clinical meaning of change in Dermatology Life Quality Index scores [abstract]. Br J Dermatol. 2002; 147(suppl 62):50. Abstract P-59; Basra M, Salek M, Camilleri L, et al. Determining the minimal clinically important difference and responsiveness of the Dermatology Life Quality Index (DLQI): further data. Dermatology. 2015; 230(1):27-33). DLQI scores have been found to correlate with other measures in observational studies, further establishing its construct validity in HE, including a significant correlation with disease severity as measured by the clinician-assessed HECSI (p<0.001) in a European study of 416 patients with HE (Agner T, Andersen K E, Brandao F M, et al. Hand eczema severity and quality of life: a cross-sectional, multicentre study of hand eczema patients. Contact dermatitis. 2008; 59(1):43-47).

Such PRO measurements may also include the Itch Numeric Rating Scale (NRS). The Itch NRS is a patient-administered, 11-point horizontal scale anchored at 0 and 10, with 0 representing “no itch” and 10 representing “worst itch imaginable”. Overall severity of a patient's itching is indicated by selecting the number that best describes the worst level of itching in the past 24 hours (Naegeli A N, Flood E, Tucker J, et al. The Worst Itch Numeric Rating Scale for patients with moderate to severe plaque psoriasis or psoriatic arthritis. Int J Dermatol. 2015; 54(6):715-722; Kimball A B, Naegeli A N, Edson-Heredia E, et al. Psychometric properties of the Itch Numeric Rating Scale in patients with moderate-to-severe plaque psoriasis. Br J Dermatol. 2016; 175(1):157-162).

In some embodiments, the treatment with baricitinib will provide that the patient has at least a 2 point improvement at the Itch Numeric Rating Scale at week 2, at week 4, at week 16, at week 32 or at some other time period. In other embodiments, treatment with baricitinib will provide that the patient has at least a 4 point improvement at the Itch Numeric Rating Scale at week 2, at week 4, at week 16, at week 32 or at some other time period. In other embodiments, treatment with baricitinib will provide that the patient has at least a 8 point improvement at the Itch Numeric Rating Scale at week 2, at week 4, at week 16, at week 32 or at some other time period.

Such PRO measurements may also include the Skin Pain NRS score. Skin Pain NRS is a patient-administered, 11-point, horizontal scale anchored at 0 and 10, with 0 representing “no pain” and 10 representing “worst pain imaginable”. Overall severity of a patient's skin pain is indicated by selecting the number that best describes the worst level of skin pain in the past 24 hours.

In some embodiments, treatment with baricitinib may cause an improvement of at least 2 points, at least 4 points, at least 6 points or at least 8 points, on the Skin Pain NRS scale when measured at one or more specific time points. These time points may be at week 2, week 4, week 6, week 8, week 10, week 12, week 14, week 16, week 18, week 20, week 22, week 24, week 26, week 28, week 30, or week 32, or at some other time point.

Such PRO measurements may also include the Hospital Anxiety Depression Scale (HADS) score. The HADS is a 14-item, self-assessment scale that determines the levels of anxiety and depression that a patient has experienced over the previous week. The HADS utilizes a 4-point Likert scale (e.g., 0 to 3) for each question and is intended for ages 12 to 65 years (Zigmond A S, Snaith R P. The hospital anxiety and depression scale. Acta Psychiatr Scand. 1983; 67(6):361-370; White D, Leach C, Sims R, et al. Validation of the Hospital Anxiety and Depression Scale for use with adolescents. Br J Psychiatry. 1999; 175(5):452-454). Scores for each domain (anxiety and depression) can range from 0 to 21, with higher scores indicating greater anxiety or depression (Zigmond A S, Snaith R P. The hospital anxiety and depression scale. Acta Psychiatr Scand. 1983; 67(6):361-370; Snaith R P. The hospital anxiety and depression scale. Health Quality Life Outcomes. 2003; 1:29).

In some embodiments, treatment with baricitinib may cause an improvement on the HADS score from baseline of 10%, 20%, 30%, 40%, 50%, 60%, 70% at one or more specific time points. These time points may be at week 2, week 4, week 6, week 8, week 10, week 12, week 14, week 16, week 18, week 20, week 22, week 24, week 26, week 28, week 30, or week 32, or at some other time point.

Such PRO measurements may also include the Work Productivity and Activity Impairment Questionnaire-Atopic Dermatitis (WPAI-AD) Scores. The WPAI-AD records impairment due to AD during the past 7 days. The WPAI-AD consists of 6 items grouped into 4 domains: absenteeism (work time missed), presenteeism (impairment at work/reduced on-the-job effectiveness), work productivity loss (overall work impairment/absenteeism plus presenteeism), and activity impairment. Scores are calculated as impairment percentages (Reilly M C, Zbrozek A S, Dukes E M. The validity and reproducibility of a work productivity and activity impairment instrument. Pharmacoeconomics. 1993; 4(5):353-365), with higher scores indicating greater impairment and less productivity.

In some embodiments, treatment with baricitinib may cause an improvement on the WPAI-AD score from baseline of 10%, 20%, 30%, 40%, 50%, 60%, 70% at one or more specific time points. These time points may be at week 2, week 4, week 6, week 8, week 10, week 12, week 14, week 16, week 18, week 20, week 22, week 24, week 26, week 28, week 30, or week 32, or at some other time point.

Such PRO measurements may also include the Atopic Dermatitis Sleep Scale (ADSS). The ADSS is a 3-item, patient-administered questionnaire developed to assess the impact of itch on sleep including: difficulty falling asleep, frequency of waking last night, and difficulty getting back to sleep. Patients rate their difficulty falling asleep and difficulty getting back to sleep, Items 1 and 3, respectively, using a 5-point Likert-type scale with response options ranging from 0 “not at all” to 4 “very difficult”. Patients report their frequency of waking, Item 2, by selecting the number of times they woke up each night, ranging from 0 to 29 times. The ADSS is designed to be completed each day with respondents thinking about sleep “last night”. Each item is scored individually.

In some embodiments, treatment with baricitinib may cause an improvement on the ADSS score from baseline of 10%, 20%, 30%, 40%, 50%, 60%, 70% at one or more specific time points. These time points may be at week 2, week 4, week 6, week 8, week 10, week 12, week 14, week 16, week 18, week 20, week 22, week 24, week 26, week 28, week 30, or week 32, or at some other time point.

It is believed that many of the retinoid side effects that are associated with current HE treatments are measured by one or more of the PRO scales (such as dry skin, sensitivity to sun, redness, itchy skin) will be obviated by treatment with baricitinib. Accordingly, the present therapy provides an improvement over the current standard of care.

Further assessments may involve analyzing or assessing itch and skin pain, specifically in the hands using visual analogue scales, as validated scales specific to the hands are not currently available.

As referred to herein, the terms “individual,” “subject,” and “patient,” used interchangeably herein, refer to a human that is suffering from HE. In a certain embodiment, the subject is further characterized with a disease, disorder, or condition that would benefit from a decreased bioactivity of JAK 1 or JAK 2.

As used interchangeably herein, “treatment” and/or “treating” and/or “treat” are intended to refer to all processes wherein there may be a slowing, interrupting, arresting, controlling, stopping, or reversing of the progression of the disorders described herein, but does not necessarily indicate a total elimination of all disorder symptoms. Treatment includes administration of baricitinib for treatment of HE includes: (a) inhibiting further progression of HE, i.e., arresting its development; and (b) relieving HE, i.e., causing regression of HE or alleviating symptoms or complications thereof. Treatment also includes preventing the onset of HE, preventing the likelihood of the onset of HE, and/or reducing the severity of HE. Treatment also includes preventing an episode or an “attack” of HE and/or reducing the likelihood that such an “attack” occurs.

In some embodiments, the patient that receives the baricitinib (e.g., the patient that has HE does not have rheumatoid arthritis, lupus or atopic dermatitis.

In some embodiments, it may be advantageous to have inclusion and/or exclusion criteria for patients receiving baricitinib for treatment of HEFor example, in some embodiments, the patient may have one or more of the following characteristics:
Male or female with an age greater than or equal to 18 years old;
Patients with moderate-to-severe chronic (>6 months) atopic HE with an HECSI greater than or equal to 17, which defines moderate HE (Oosterhaven and Schuttelaar 2020, Responsiveness and interpretability of the Hand Eczema Severity Index. Br J Dermatol. 2020; 182(4), 932-939);
   For atopic HE, inadequate response or intolerance to greater than or equal to 1 high-potency TCS <6 months prior to screening;
Moderate-to-severe AD for greater than or equal to 12 months defined by an IGA of greater than or equal 3;
   Greater than or equal to 1% BSA; and
   EASI >7.

In additional embodiments, the key inclusion criteria may be as follows:

Patients will be required to have moderate-to-severe AD as defined by a validated IGA score of 3 or 4 and to be candidates for systemic treatment. An inclusion criterion stipulating a current diagnosis of atopic HE will be added. Importantly, patients whose contact dermatitis on the hands is the result of known exogenous trigger(s) may not fully benefit from the present treatment, and thus, may not be administered the treatment. This will limit the treatment to those where the HE has taken a chronic course and where avoidance of the trigger alone does not lead to eczema resolution. Additionally, the following inclusion criteria may also be used: the allowance of up to approximately 40% of the trial population to have less than or equal to 10% of BSA affected, and the baseline EASI cutoff score is lowered from 16 to >7.

In certain embodiments, key exclusion criteria may be as follows:
Patients who, in the assessment by the investigator, have clinically relevant allergic contact dermatitis of the hands;
Patients with confirmed exposure to irritants believed to be a predominant cause of the current HE;
Patients who are aware of an upcoming occupational or lifestyle change that could potentially affect their underlying condition during the duration of the trial.

It has been reported that two patients with HE have been successfully treated with baricitinib (4 mg tablet once per day) (F. M. Rosenberg, et al., Baricitinib Treatment of Severe Chronic Hand Eczema: Two Case Reports—2022—Contact Dermatitis—Wiley Online Library).

In the first case, a 52-year-old man, who works as a builder, was diagnosed with severe hyperkeratotic HE for 6 years. He has a history of asthma in childhood, allergic rhino-conjunctivitis, and no history of AD. This patient had a ‘severe’ HE and a hand eczema severity index (HECSI) score of 55 (severe) at the start of treatment. After 16 weeks of treatment, the HE was improved to “almost clear” and the HECSI score to 4. The patient's quality of life improved from “strongly impaired” to “not at all impaired” based on the Quality of Life in Hand Eczema Questionnaire (QOLHEQ). Emollients were continued during baricitinib treatment. Baricitinib was well-tolerated as no side effects occurred.

In the second case, a 55-year-old woman, who works as an administrator, had severe AD with concomitant severe atopic HE for 5 years. She has a history of allergic asthma, allergic rhino-conjunctivitis, food allergies, and kerato-conjunctivitis. She had no relevant exposure to irritants, and there were no contact allergies with clinical relevance to the hands. This patient had a ‘severe’ HE and a hand eczema severity index (HECSI) score of 47 (severe) at the start of treatment.

Baricitinib dosage was tapered to 2 mg per day at 12 weeks on request of the patient due to the good effect. After 16 weeks of treatment, the HE was improved to “almost clear” and the HECSI score to 8. Her quality of life improved from “moderately impaired” to “not at all impaired” based on the QOLHEQ. However, she discontinued baricitinib because of a bacterial corneal ulcer at 16 weeks.

### EXAMPLE 1—PROPHETIC IN VIVO STUDY

Patients will be divided into treatment groups consisting of double-blinded placebo and baricitinib therapy groups. Baricitinib therapy groups will be administered an amount of baricitinib (for example, a 4 mg pill or tablet in the manner outlined herein), whereas the placebo group are administered a pill that has only placebo (e.g., a 4 mg pill or tablet of placebo).

There will be generally 3 periods in the study, e.g., a “screening period,” a “treatment period” and a “follow-up period”. All patients go through each of these periods.

In the screening period, which occurs before treatment begins, the patients will be assessed for one or more of the following:
the patient's HECSI score;
the patient's EASI score;
the patient's NRS itch score;
the vIGA-AD score;
the patient's HADS score;
the patient's HRQoL score;
the patient's IGA (investigator Global Assessment) score;
the patient's mTLSS score;
the patient's WPAI score; and
   During this screening period, which is usually for about 4 weeks prior to Day 0, the patients will stop receiving treatments for HE (as a way of “washing out” prior treatments). However, starting about 1 week before Day 0, the patients can use TCS.

At Day 0, the patient will receive his/her first treatment of baricitinib or placebo depending upon which group into which they are assigned. However, prior to administering baricitinib or placebo, measurements of the above-recited scores will be obtained (most especially the HECSI score).

During the treatment period, the patients will be given either placebo or baricitinib (depending upon which group of the study they are in). In some situations, randomized patients will take the first dose of investigational product at the clinic and pharmacokinetic (PK) samples will be drawn 15 minutes and 1 hour post dose. Baricitinib may be daily dosed for 16 weeks. Clinical assessments and laboratory samples, including additional PK sampling, is obtained at scheduled visits during the treatment period. During the treatment period, in addition to randomized treatment, patients will also maintain TCS. Re-assessment of the patient's measurements (such as those measurements above) may occur at any time during the treatment period.

After the treatment period (which may last, for example, 16 weeks), the patient enters the follow-up period. During this period, the patient's measurements are re-assessed (or further re-assessed), including the scores noted above. This additional treatment may last for an additional 16 weeks (for a total of 32 weeks). During this additional 16-week period, those on placebo may be switched over to baricitinib treatment.

### EXAMPLE 2

In collaboration with Davos Biosciences GmbH, clinical studies have been conducted using baricitinib for treating HE at a daily dosage of 4 mg. Photographic images and additional clinical data demonstrate that baricitinib was effective for this indication.

1. A method of treating a patient in need of treatment for hand eczema, comprising administering to said patient an amount of baricitinib, or a pharmaceutically acceptable salt or formulation thereof.

2. The method of claim 1, wherein the amount of baricitinib, or the pharmaceutically acceptable salt or formulation thereof, is administered orally.

3. The method of claim 1, further comprising:
assessing the patient's HECSI score at Day 0 before administration of baricitinib; and
re-assessing the patient's HECSI score following the step of administering baricitinib.

4. The method of claim 3, wherein the patient's HECSI score is re-assessed after 16 weeks of receiving a daily administration of baricitinib, wherein the HECSI score value assessed after week 16 is at least 75% lower than the HECSI score assessed on Day 0.

5. The method of claim 1, further comprising:
assessing the patient's EASI score at Day 0; and
re-assessing the patient's EASI score following the step of administering baricitinib daily for 16 weeks.

6. The method of claim 1, further comprising:
assessing the patient's score at Day 0 in one or more of the following tests:
the patient's NRS itch score;
the vIGA-AD score;
the patient's HADS score;
the patient's HRQoL score;
the patient's IGA score;
the patient's mTLSS score;
the patient's WPAI score; and
re-assessing the patient's score in those tests following the step of administering baricitinib daily for 16 weeks.

7. The method of claim 1, wherein the daily dose of baricitinib is 4 mg.

8. Use of baricitinib in the manufacture of a medicament for the treatment of hand eczema.

9-13. (canceled)

14. The method of claim 1, wherein the patient meets one or more of the following criteria prior to administration:
a HECSI score greater than to equal to 17,
an IGA score of greater than or equal 3;
greater than or equal to 1% BSA affected with HE; and
an EASI score greater than or equal to 7.

15. The method according to claim 1, wherein the patient meets the following criteria prior to administration:
a HECSI score greater than to equal to 17.

16. The method according to claim 1, wherein the patient meets the following criteria prior to administration:
an IGA score of greater than or equal 3.

17. The method according to claim 1, wherein the patient meets the following criteria prior to administration:
greater than or equal to 1% BSA affected with HE.

18. The method according to claim 1, wherein the patient meets the following criteria prior to administration:
an EASI score greater than or equal to 7.

19. The method of claim 1, wherein the administering is in the form of a pill.

20. The method of claim 19, wherein the administering is in the form of one or more 4 mg pill(s).

21. The method of claim 19, wherein the administering is in the form of one or more 4 mg pill(s) each including one or more excipients.

22. The method of claim 1, wherein the administering is administering of a formulation of baricitinib.

23. The method of claim 1, wherein the administering is administering of a formulation of a pharmaceutically acceptable salt baricitinib.

24. A method of treating a patient in need of treatment for hand eczema comprising administering to said patient baricitinib in the form of a 4 mg pill once a day.

25. The use of claim 8, wherein the medicament is in the form of a 4 mg pill.
